(19) United States
(12) Patent Application Publication
(10) Pub. No.: US 2023/0416293 A1
(43) Pub. Date: Dec. 28, 2023

(54) SYNTHESIS OF [18F]-LABELED THYMIDINE ANALOGUES

(71) Applicant: UNIVERSITY OF SOUTHERN CALIFORNIA, Los Angeles, CA (US)

(72) Inventors: Kai CHEN, Los Angeles, CA (US); Peter S. CONTI, Los Angeles, CA (US)

(73) Assignee: UNIVERSITY OF SOUTHERN CALIFORNIA, Los Angeles, CA (US)

(21) Appl. No.: 18/253,857

(22) PCT Filed: Nov. 19, 2021
(86) PCT No.: PCT/US21/60050

§ 371 Date: May 22, 2023

Related U.S. Application Data
(60) Provisional application No. 63/117,192, filed on Nov. 23, 2020.

(51) Int. Cl. C07H 19/09; A61K 51/04

(52) U.S. Cl. CPC C07H 19/09; A61K 51/0491

(57) ABSTRACT

Thymidine analogues, 5-substituted 2′-deoxy-2′-[¹⁸F]fluoro-arabinofuranosyluracil derivatives, are promising positron emission tomography (PET) tracers being evaluated for noninvasively imaging cancer cell proliferation and/or reporter gene expression. We report the radiosynthesis of 2′-deoxy-2′-[¹⁸F]fluoro-5-methyl-1-β-d-arabinofuranosyluracil ([¹⁸F]FMAU) and other 2′-deoxy-2′-[¹⁸F]fluoro-5-substituted-1-β-d-arabinofuranosyluracil analogues using 1,4-dioxane to replace the currently used 1,2-dichloroethane. Compared to 1,2-dichloroethane, 1,4-dioxane is analyzed as a better solvent in terms of radiosynthetic yield and toxicity concern. The use of a less toxic solvent allows for the translation of the improved approach to clinical production. The new radiolabeling method can be applied to an extensive range of uses for ¹⁸F-labeling of other nucleoside analogues.

### RELATED APPLICATIONS

This application claims priority under 35 U.S.C. § 119(e) to U.S. Provisional Patent Application No. 63/117,192, filed Nov. 23, 2020, which is incorporated herein by reference in its entirety.

### BACKGROUND OF THE INVENTION

Excessive cellular proliferation is one of many distinct cancer-related hallmarks. A good number of extra-organismal assays have been developed to measure tumor proliferation rates. However, these assays largely require invasive procedures to remove a small piece of living tissues or a sample of cells from the body, rendering difficulties in assessing tumor proliferation in a real time, over the course of treatment, and in multiple regions, particularly for patients with diverse metastatic lesions. Molecular imaging has emerged at the forefront in the area of “personalized medicine” to obtain timely and noninvasive evaluation of biological and physiological processes in living bodies and improve our understanding of diseases. Radiofluorinated analogues of 2′-deoxy-2′-fluoro-5-substituted-1-β-D-arabinofuranosyluracil (FIG. 1) are promising PET radiotracers for evaluating tumor proliferation and imaging reporter gene expression. The radiotracers are phosphorylated by thymidine kinases TK1 and/or TK2 and further integrated into host DNA (FIG. 1). There is evidence that a therapeutic response could be defined earlier and perhaps more accurately by measuring changes in DNA synthesis within tumors. Therefore, 2′-deoxy-2′-[¹⁸F]fluoro-5-substituted-1-β-D-arabinofuranosyluracil analogues have great potential for use in not only early diagnosis of diseases, but also identifying treatment effects, and thus assisting in the clinical decision-making process and enabling treatment optimization for individual patients (“personalized medicine”).

¹⁸F is one of the most common radionuclides for PET imaging because of its excellent chemical and nuclear-physical properties. ¹⁸F has a half-life of 109.77 min which allows multistep synthesis and longer imaging protocols. In addition, the low β⁺ energy of 18F, 0.64 MeV, leads to high-resolution PET images due to a short positron linear range in tissue. 2′-Deoxy-2′-[¹⁸F]fluoro-5-methyl-1-β-D-arabinofuranosyluracil ([¹⁸F]FMAU) is a promising PET tracer currently being investigated in preclinical studies and clinical trials for evaluating cell proliferation in multiple carcinomas, such as breast carcinoma, prostatic carcinoma, and non-small cell lung carcinoma. An advantage of [¹⁸F]FMAU, over another widely used thymidine analogue 3′-deoxy-3′-[¹⁸F]fluorothymidine ([¹⁸F]FLT), is the ability to incorporate [¹⁸F]FMAU into DNA. Compared with [¹⁸F]FMAU, [¹⁸F]FLT cannot be substantially incorporated into DNA due to the fluorinated 3′-position of deoxyribose acting as a terminator of the growing DNA chain. In addition, 2′-deoxy-2′-[¹⁸F]fluoro-5-ethyl-1-β-D-arabinofuranosyluracil ([¹⁸F]FEAU) and 2′-deoxy-2′-[¹⁸F]fluoro-5-iodo-1-β-D-arabinofuranosyluracil ([¹⁸F]FIAU) are PET radiotracers for imaging reporter gene herpes virus type 1 thymidine kinase (HSV1-tk) expression. Therefore, they have been used for gene-based therapy, transgenic models, and cell trafficking. 2′-Deoxy-2′-[¹⁸F]fluoro-1-β-D-arabinofuranosyluracil ([¹⁸F]FAU) can be phosphorylated and methylated by thymidine kinase and thymidylate synthase respectively, and then incorporated into DNA. Consequently, [¹⁸F]FAU is a promising PET probe for evaluating tumors growth and studying the pharmacokinetics and metabolism of FAU acting as a chemotherapeutic agent. In addition, 2′-deoxy-2′-[¹⁸F]fluoro-5-fluoro-1-β-D-arabinofuranosyluracil ([¹⁸F]FFAU) and 2′-deoxy-2′-[¹⁸F]fluoro-5-chloro-1-β-D-arabinofuranosyluracil ([¹⁸F]FCAU) are also promising PET probes for imaging the expression of HSV1-tk genes.

Radiolabeling of [¹⁸F]FMAU and its thymidine analogues, involving the radiosynthesis of 2-[¹⁸F]fluoro-1,3,5-tri-O-benzoyl arabinofuranose and its conversion to 1-bromo-2-[¹⁸F]fluoro-1,3,5-tri-O-benzoyl arabinofuranose. The latter could be coupled to various 2,4-bis-trimethylsilyluracil derivatives. Hydrolysis of the protecting groups from the sugar moiety provided the desired products. However, this method of making 2′-deoxy-2′-fluoro-5-substituted-1-β-D-arabinofuranosyluracil analogues is rather tedious, involving multi-step procedures leading to a low radiochemical yield of desired products and inconvenience for clinical use. The synthetic approach using Friedel-Crafts catalysts was previously reported to simplify synthesis conditions and shorten reaction time. However, a very toxic solvent, 1,2-dichloroethane (DCE), was employed as the solvent in the coupling of 2-deoxy-2-[¹⁸F]fluoro-1,3,5-tri-O-benzoyl-D-arabinofuranose (¹⁸F-labeled sugar) and uracil bases. In the United States Pharmacopeia (USP) General Chapter <467>, DCE is defined as a Class 1 residual solvent and its injectable concentration limits at 5 parts per million (ppm) due to its highly toxic potential to humans. The residual DCE in the PET drug injection is strictly controlled by the US Food and Drug Administration (FDA). In addition, the quantitation limit of extremely low concentration of residual solvents, such as DCE (≤5 ppm), puts forward a huge challenge on the method validation of gas chromatography. Therefore, the finding of a suitable solvent for the radiosynthesis of [¹⁸F]FMAU and its analogues is in urgent demand for paving the way for their clinical translation. The present disclosure satisfies this need.

### SUMMARY OF THE INVENTION

The present disclosure relates to compositions and methods of synthesizing 2′-deoxy-2′-[¹⁸F]-fluoro-5-substituted-1-β-D-arabinofuranosyl-uracil and cytosine compounds in a one-pot reaction. The method comprises radiolabeling a precursor sugar with ¹⁸F, contacting the ¹⁸F radiolabeled sugar with a silylated uracil or cytosine in the presence of 1,4-dioxane, trimethylsilyl trifluoromethanesulfonate (TMSOTf), and hexamethyldisilazane (HMDS), incubating the components under conditions that allow for conjugation of the ¹⁸F radiolabeled sugar and the silylated uracil or cytosine, and removing the protecting groups of the components. The synthesis may take place in a fully automated cGMP-compliant radiosynthesis module.

In some embodiments, the invention relates to compositions and methods of synthesizing [¹⁸F]-labeled 2′-deoxy-arabino 5-substituted or unsubstituted uracil or cytosine nucleoside in a one-pot reaction. The method comprises radiolabeling of a precursor sugar with ¹⁸F, contacting the ¹⁸F radiolabeled sugar with a silylated uracil or cytosine in the presence of 1,4-dioxane, TMSOTf, and HMDS, incubating the components under conditions that allow for conjugation of the ¹⁸F radiolabeled sugar and the silylated uracil or cytosine, and removing the protecting groups of the components.

Additional embodiments relate to methods of synthesizing 2′-deoxy-2′-[¹⁸F]-fluoro-5-substituted-1-β-D-arabinofuranosyl-uracil or cytosine compounds in a one-pot reaction. The one-pot synthesis reaction includes radiolabeling of a precursor sugar with ¹⁸F, filtering the ¹⁸F radiolabeled sugar through a cartridge (e.g., ion exchange cartridge), contacting the ¹⁸F radiolabeled sugar with a silylated uracil or cytosine in the presence of a Friedel-Crafts catalyst and 1,4-dioxane, incubating the components under conditions that allow for conjugation of the ¹⁸F radiolabeled sugar and the silylated uracil or cytosine, and removing the protecting groups of the components.

In some embodiments, the [¹⁸F]-labeled thymidine or cytidine analogue can be used as a probe for imaging tumor proliferative activity. These [¹⁸F]-labeled thymidine or cytidine analogue can be used as a PET tracer for certain medical conditions, including, but not limited to, cancer disease, autoimmunity inflammation, and bone marrow transplant.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Chemical structures of 2′-deoxy-2′-[¹⁸F]fluoro-5-substituted-1-β-D-arabinofuranosyluracil analogues and their involvement in potential DNA synthesis pathways. The R group is a hydrogen, methyl, ethyl, fluorine, chlorine, bromine, or iodine, and the radiotracer compound is [¹⁸F]FAU, [¹⁸F]FMAU, [¹⁸F]FEAU, [¹⁸F]FFAU, [¹⁸F]FCAU, [¹⁸F]FBAU, [¹⁸F]FIAU, respectively.

FIG. 2. Analytical HPLC profiles of crude product in the radiosynthesis of [¹⁸F]FMAU using polar solvents: dimethyl sulfoxide (DMSO), N,N-dimethylformamide (DMF), tetrahydrofuran (THF), and non-polar solvents: 1,2-dichloroethane (DCE), 1,4-dioxane. Arrows indicate the desired [¹⁸F]FMAU product (β-anomer).

FIG. 3. Analytical HPLC profiles of the crude product (A1-G1) and the final product (A2-G2) in the radiosynthesis of [¹⁸F]FAU, [¹⁸F]FMAU, [¹⁸F]FEAU, [¹⁸F]FFAU, [¹⁸F]FCAU, [¹⁸F]FBAU, and [¹⁸F]FIAU using 1,4-dioxane as the solvent. The peaks labeled with retention time indicate the desired product (β-anomer).

FIG. 4. Analytical HPLC profile of crude [¹⁸F]FMAU product using the protected thymine (O,O′-bis(trimethylsilyl)thymine) or thymine and 1,4-dioxane in the coupling step at different reaction times and temperatures. Arrows indicate the desired [¹⁸F]FMAU product (β-anomer).

FIG. 5. Coupling efficiency of 2-deoxy-2-[¹⁸F]fluoro-1,3,5-tri-O-benzoyl-D-arabinofuranose (¹⁸F-labeled sugar) and the protected thymine (O,O′-bis(trimethylsilyl)thymine) or thymine using 1,4-dioxane as the solvent at different reaction times and temperatures: (A) Radiochemical yield (%) based on analytical HPLC; (B) Ratio of anomers (β/α) at 60 min. Statistical significance between two groups is shown (*P<0.05; **P<0.01; NS, non-significant).

FIG. 6. Representative microPET images of subcutaneous MDA-MB-231 (A1-A4) and U-87 MG (B1-B4) tumor-bearing nude mice at 1 and 2 h post-injection (p.i.) of [¹⁸F]FMAU. Tumor-to-muscle (T/M) ratio, tumor-to-liver (T/L) ratio, and tumor-to-kidney (T/K) ratio of [¹⁸F]FMAU at 1 h and 2 h p.i. with mouse xenograft models bearing subcutaneous MDA-MB-231 (C) or U-87 MG (D) tumor. Arrows indicate tumors. A heat map represents a scale of 8.0% ID/g to 0.2% ID/g where the highest accumulation of [18^{F}]FMAU is in white and the lowest accumulation of [¹⁸F]FMAU is in black.

FIG. 7. Schematic of radiosynthesis module for the ¹⁸F labeling of thymidine analogues.

FIG. 8. Semi-preparative HPLC UV (A) and radioactivity (B) of crude [¹⁸F]FMAU product.

FIG. 9. Analytical HPLC UV (A) and radioactivity (B) for co-injection of cold authentic anomers (α- and β-anomer) and [¹⁸F]FMAU.

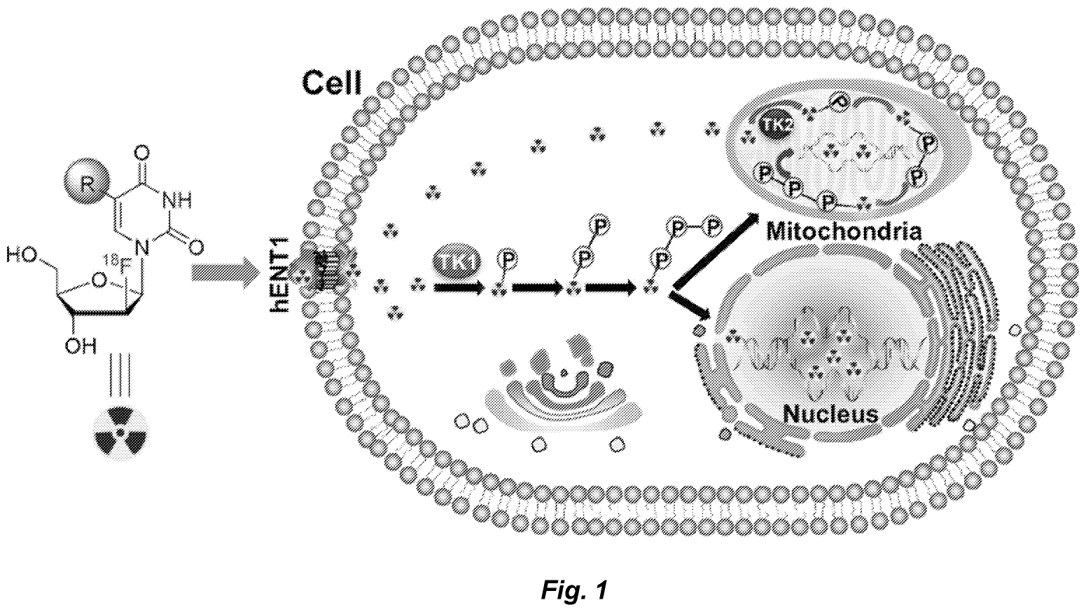

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R.J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number 10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

The term “one-pot” is a term commonly used by ordinary persons skilled in the art referring to a strategy to improve the efficiency of a chemical reaction whereby a reactant is subjected to successive chemical reactions in just one reactor. The strategy avoids a lengthy separation and purification steps of intermediate chemical compounds and saves time and resources while increasing chemical yield. A one-pot synthesis may require changing a solvent to a different solvent at one or more steps during the procedure, for example, by simply evaporation under reduced pressure. Alternatively, it may be possible to perform the synthesis with a single suitable solvent that can be used throughout the entire procedure without changing the solvent. Generally, a sequential one-pot synthesis is performed by adding reagents to a reactor one at a time and without work-up.

### Embodiments of the Invention

The feasibility of using polar and nonpolar solvents in coupling of ¹⁸F-labeled sugar and 5-substituted uracil using trimethylsilyl trifluoromethanesulfonate (TMSOTf) and hexamethyldisilazane (HMDS) (Scheme 1) was studied. After the unexpected and surprising identification of 1,4-dioxane as a solvent, the synthetic conditions were adjusted, including reaction temperature and time, in the coupling step to enhance the overall radiolabeling yield and ratio of anomers (β/α). The newly developed method was applied for the radiosynthesis of 2′-deoxy-2′-[¹⁸F]fluoro-5-substituted-1-β-D-arabinofuranosyluracil analogues to show scope of the synthesis method and the resulting [¹⁸F]FMAU tracers were then subjected to microPET imaging of tumor-bearing mice.

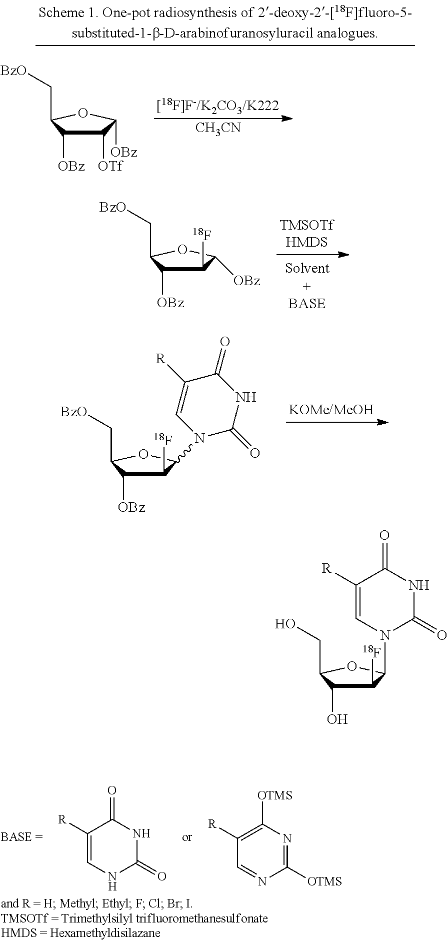

Embodiments of the disclosure provide methods of synthesizing 2′-deoxy-2′-[¹⁸F]-fluoro-5-substituted-1-β-D-arabinofuranosyl-uracil or cytosine compounds in a one-pot reaction comprising: a) radiolabeling a precursor sugar with ¹⁸F; b) contacting the ¹⁸F radiolabeled sugar with a silylated uracil or cytosine in the presence of 1,4-dioxane, a Friedel Crafts catalyst such as trimethylsilyl trifluoromethanesulfonate (TMSOTf), and hexamethyldisilazane (HMDS); c) incubating the components in step (b) under conditions that allow for conjugation of the ¹⁸F radiolabeled sugar and the silylated uracil or cytosine; d) removing the protecting groups of the components in step (c); and optionally e) purifying the deprotected product. Preferably, the 2′-deoxy-2′-[¹⁸F]-fluoro-5-substituted-1-β-D-arabinofuranosyluracil is 2′-deoxy-2′-[¹⁸F]fluoro-5-methyl-1-β-D-arabino-furanosyl-uracil ([¹⁸F]FMAU).

In some embodiments, a combination of solvents is used in step (b). In other embodiments, the solvent used in step (b) consists essentially of 1,4-dioxane, or consists of 1,4-dioxane (i.e., is the only solvent used in step (b)).

In some embodiments, the solvents of the reaction include one or more of 1,4-dioxane, a Friedel Crafts catalyst such as trimethylsilyl trifluoromethanesulfonate (TMSOTf), and hexamethyldisilazane (HMDS). Preferably, the solvent does not contain 1,2-dichloroethane.

In some embodiments, the solvents of the reaction comprise 1,4-dioxane, a Friedel Crafts catalyst such as trimethylsilyl trifluoromethanesulfonate (TMSOTf), and hexamethyldisilazane (HMDS). Preferably, the solvent does not contain 1,2-dichloroethane.

In some embodiments, the solvents of the reaction comprise 1,4-dioxane with the proviso that the solvent does not contain 1,2-dichloroethane.

In some embodiments, the solvents of the reaction consist essentially of 1,4-dioxane, a Friedel Crafts catalyst such as trimethylsilyl trifluoromethanesulfonate (TMSOTf), and hexamethyldisilazane (HMDS), or consists essentially of 1,4-dioxane.

As used herein, “a Friedel-Crafts catalyst” refers to any catalyst required for a Friedel-Crafts reaction. Friedel-Crafts reaction are a set of reactions developed by Charles Friedel and James Crafts in 1877 to attach substituents to an aromatic ring. Friedel-Crafts reactions are of two main types: alkylation reactions and acylation reactions. Both proceed by electrophilic aromatic substitution. Examples of Friedel-Crafts catalyst include, but are not limited to trimethyl silyl trifluoromethanesulfonate, AlCh, SnCl₄, and ZnCl₂. See, for example, U.S. Pat. Publication No. US20210009624 to Chen et al., incorporated herein by reference in its entirety.

In one embodiment, the Friedel-Crafts catalyst is trimethyl silyl trifluoromethanesulfonate (TMSOTf).

In other embodiments, the method of synthesizing an [¹⁸F]-labeled 2′-deoxy-arabino-5-substituted or unsubstituted uracil or cytosine nucleoside in a one-pot reaction comprises: a) radiolabeling a precursor sugar with ¹⁸F; b) contacting the ¹⁸F radiolabeled sugar with a silylated uracil or cytosine in the presence of 1,4-dioxane, trimethylsilyl trifluoromethanesulfonate (TMSOTf), and hexamethyldisilazane (HMDS); c) incubating the components in step (b) under conditions that allow for conjugation of the ¹⁸F radiolabeled sugar and the silylated uracil or cytosine derivatives; d) removing the protecting groups of the components in step (c); and e) optionally purifying the deprotected product.

Preferably, the [¹⁸F]-labeled 2′-deoxy-arabino-5-substituted or unsubstituted uracil or cytosine nucleoside is selected from the group consisting of 2′-fluoro-5-ethyl-1-β-D-arabinofuranosyluracil (FEAU), 2′-deoxy-2′-fluoro-5-fluoro-1-β-D-arabinofuranosyluracil (FFAU), 1-(2-deoxy-2-fluoro-β-D-arabinofuranosyl)-5-chlorouracil (FCAU), 1-(2-deoxy-2-fluoro-β-D-arabinofuranosyl)-5-bromouracil (FBAU), 1-(2-deoxy-2-fluoro-(3-D-arabinofuranosyl)uracil (FAU), 2′-fluoro-2′-deoxy-1-β-D-arabinofuranosyl-5-iodouracil (FIAU), 1-(2-deoxy-2-fluoro-β-D-arabinofuranosyl)cytosine (FAC), 2′-deoxy-2′-fluoro-5-methyl-1-β-D-arabinofuranosylcytosine (FMAC), 2′-fluoro-5-ethyl-1-β-D-arabinofuranosyl-cytosine (FEAC), 2′-deoxy-2′-fluoro-5-fluoro-1-β-D-arabinofuranosyluracil (FFAC), 1-(2-deoxy-2-fluoro-β-D-arabinofuranosyl)-5-chlorocytosine (FCAC), 1-(2-deoxy-2-fluoro-β-D-arabinofuranosyl)-5-bromocytosine (FBAC), and 2′-deoxy-2′-fluoro-5-hydroxymethyl-1-β-D-arabino-furanosylcytosine (FHMAC).

In some embodiments, the method for the synthesis of [¹⁸F]-labeled thymidine or cytidine analogues occurs in a fully automated cGMP-compliant radiosynthesis module.

In another embodiment, the method of synthesizing 2′-deoxy-2′-[¹⁸F]-fluoro-5-substituted-1-β-D-arabinofuranosyl-uracil or cytosine compounds in a one-pot reaction comprises: a) radiolabeling a precursor sugar with ¹⁸F; b) filtering the ¹⁸F radio labeled sugar produced in step (a) through a cartridge; c) contacting the ¹⁸F radiolabeled sugar with a silylated uracil or cytosine in the presence of a Friedel-Crafts catalyst and 1,4-dioxane; d) incubating the components in step (c) under conditions that allow for conjugation of the ¹⁸F radiolabeled sugar and the silylated uracil or cytosine; e) incubating the components in step (d) under conditions that allow for removal of the protecting groups of the components in step (d) thereby removing the protecting groups of the components in step (d); and f) optionally purifying the deprotected product.

In some embodiments, the method further includes, before purifying the synthesized compound, via, for example, high-pressure liquid chromatography (HPLC), incubating the mixture containing the compound with sodium methoxide and methanol to remove benzoyl groups. In other aspects, the method further includes adding a carrier, excipient, diluent, or a combination thereof to the purified compound.

The [¹⁸F]-labeled thymidine or cytidine analogues disclosed herein can be used as a PET tracer for certain medical conditions, including, but not limited to, cancer disease, autoimmunity inflammation, and bone marrow transplant.

The term “cancer” refers to a group of diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure to environmental pollutant, tobacco and or alcohol use, obesity, poor diet, lack of physical activity or any combination thereof. “Metastasis” refers to the biological process involved in the development of metastases. “Neoplasm” or “tumor” including grammatical variations thereof means new and abnormal growth of tissue, which may be benign or cancerous.

Exemplary cancers include breast cancer, non-small cell lung cancer, brain cancer, and osteosarcoma. Exemplary cancers also include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland'Cancer, Childhood; Sarcoma, Ewing's Family of Tumors; Sarcoma, Kaposi's; Sarcoma (Osteosarcoma) Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

“Cancer cell” or “tumor cell”, and grammatical equivalents refer to the total population of cells derived from a tumor or a pre-cancerous lesion, including both non tumorigenic cells, which comprise the bulk of the tumor population, and tumorigenic stem cells (cancer stem cells).

As used herein, “PET” or “PET-scan” refers to positron emission tomography (PET) scanning using a molecular tracer. PET-scan is a nuclear medicine functional imaging technique that is widely used in the medical field to observe metabolic processes in the body as an aid to the diagnosis of disease.

The compounds can be administered in various modes, e.g., orally, topically, or by injection. In some embodiments, the compounds (e.g., [¹⁸F]FMAU) are administrated by injection or intravenously.

The terms “administration of” and “administering a” compound should be understood to mean providing a compound of the disclosure or pharmaceutical composition to a subject. An exemplary administration route is intravenous administration. In general, administration routes include but are not limited to intracutaneous, subcutaneous, intravenous, intraperitoneal, intraarterial, intrathecal, intracapsular, intraorbital, intracardiac, intradermal, transdermal, transtracheal, sub cuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration. The compositions of the present invention may be processed in a number of ways depending on the anticipated application and appropriate delivery or administration of the pharmaceutical composition. For example, the compositions may be formulated for injection.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### Results and Discussion

### Radiosynthesis of [¹⁸F]FMAU and its Analogues

Selection of appropriate solvents in PET drug manufacture is of great importance for translating PET drugs into clinical use. In our previous effort of radiosynthesizing 2′-deoxy-2′-[¹⁸F]fluoro-5-substituted-1-β-D-arabinofuranosyluracil analogues, we found that DCE can be used in the step of coupling ¹⁸F-labeled sugar and 5-substituted uracil, where the reaction was heated at 85° C. for 1 h to provide a β/α anomer ratio of 1.24:1 for the [¹⁸F]FMAU synthesis (Table 1). However, DCE is listed as a Class 1 residual solvent in the USP, which is known to be highly toxic to humans and thus, is difficult to use in drug manufacturing for clinical investigations. With a goal of further improving the radiosynthesis of 2′-deoxy-2′-[¹⁸F]fluoro-5-substituted-1-β-D-arabinofuranosyluracil analogues and facilitating their clinical translation, we attempted to explore other solvents.

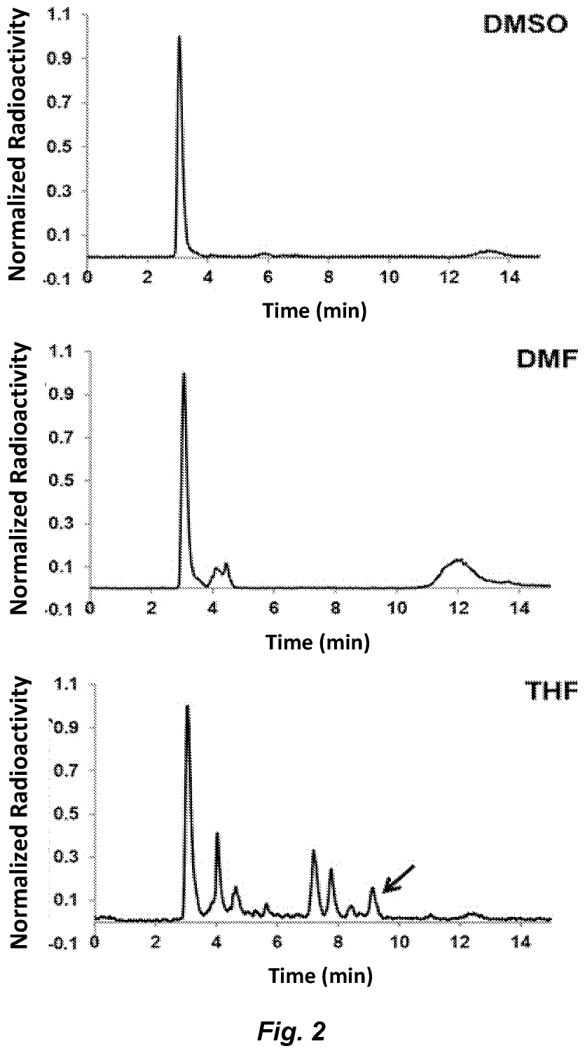

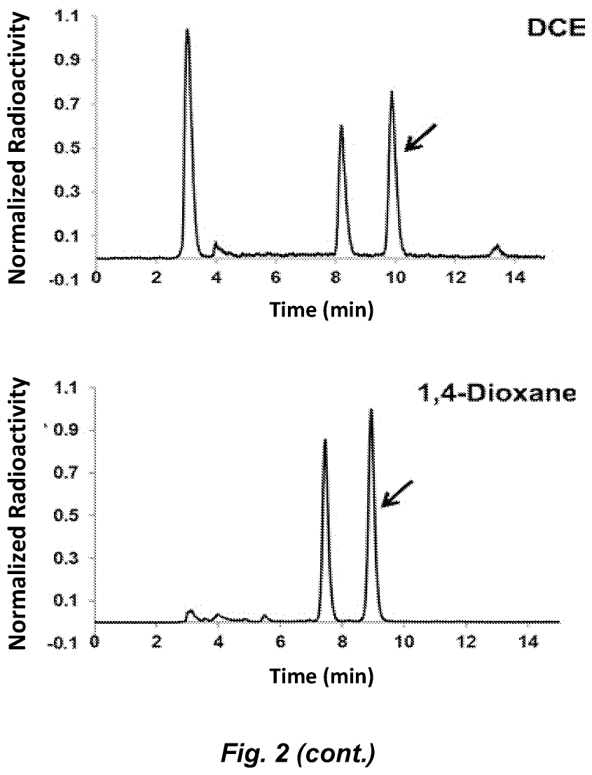

The present investigation started with some polar solvents, such as dimethyl sulfoxide (DMSO) and N,N-dimethylformamide (DMF). Interestingly, no desired products were observed using these polar solvents (Table 1 and FIG. 2). After moving to nonpolar solvents, it was found that the use of tetrahydrofuran (THF) can yield the desired products, but the radiochemical yield is minimal and unacceptable. Our continued efforts led to the identification of 1,4-dioxane, which is listed in Class II residual solvents with a residual concentration limit of 380 ppm. As compared to DCE with 5 ppm concentration limit, 1,4-dioxane is considered a greener solvent. In addition, the employment of 1,4-dioxane as compared to DCE in the radiosynthesis of [¹⁸F]FMAU afforded an improved radiochemical yield (RCY) of the desired β-anomer product (48.07% vs. 32.68%) (Table 1 and FIG. 2).

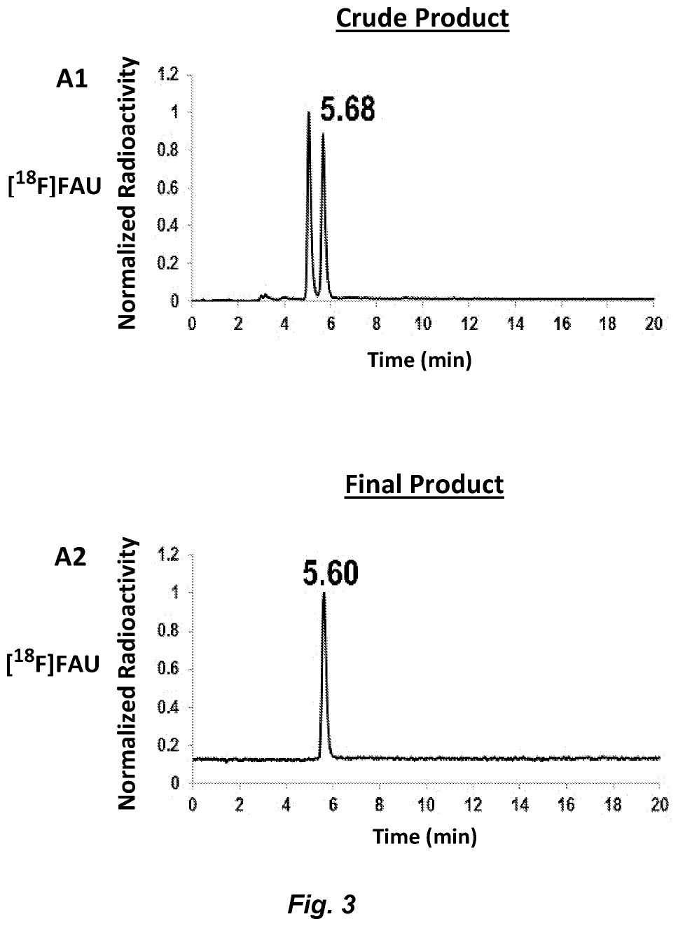

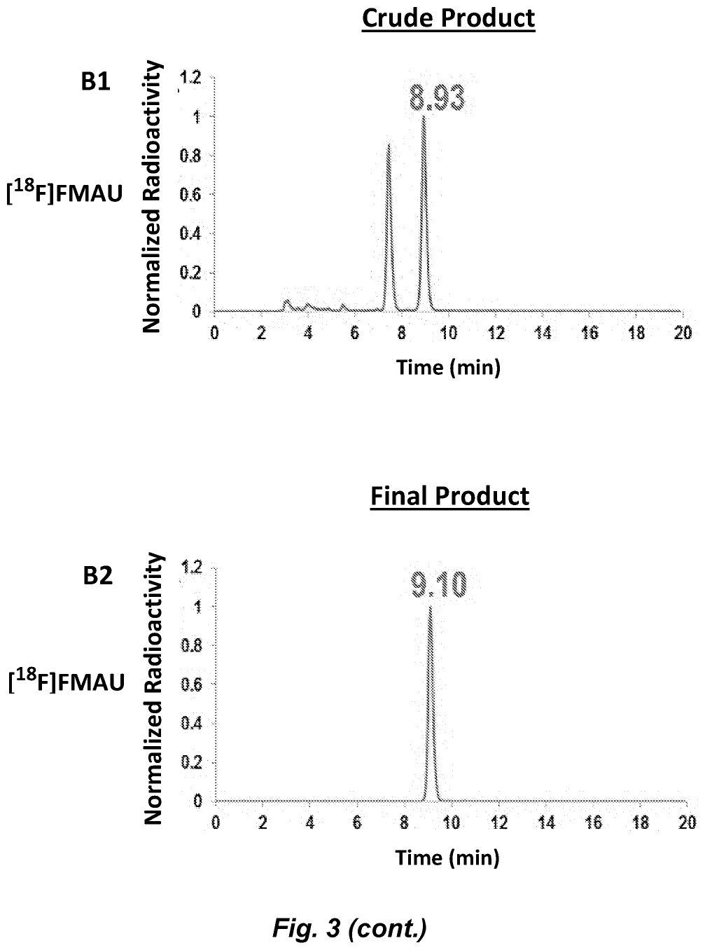

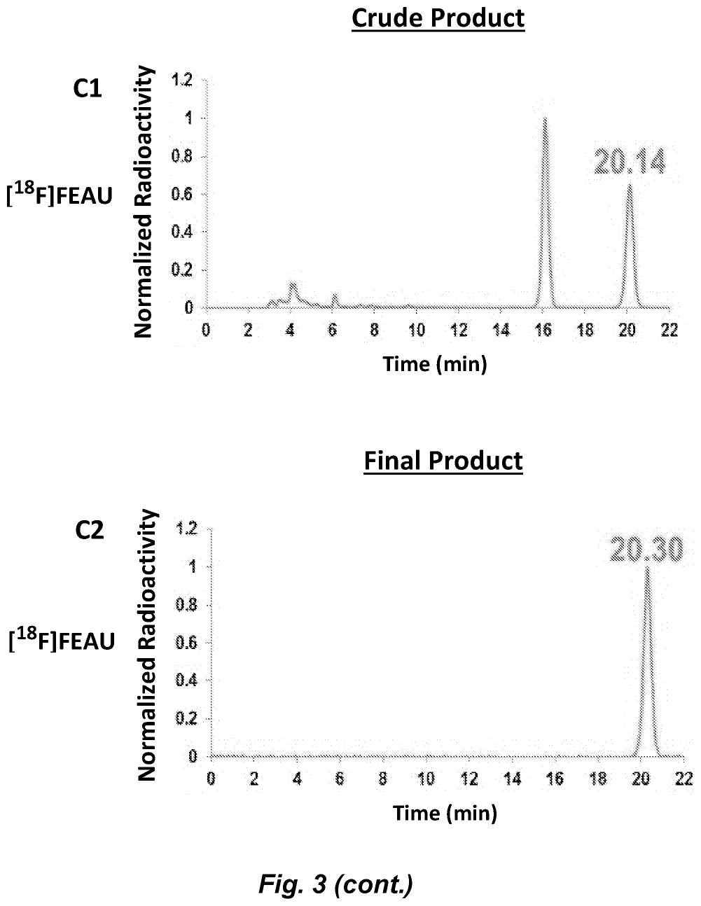

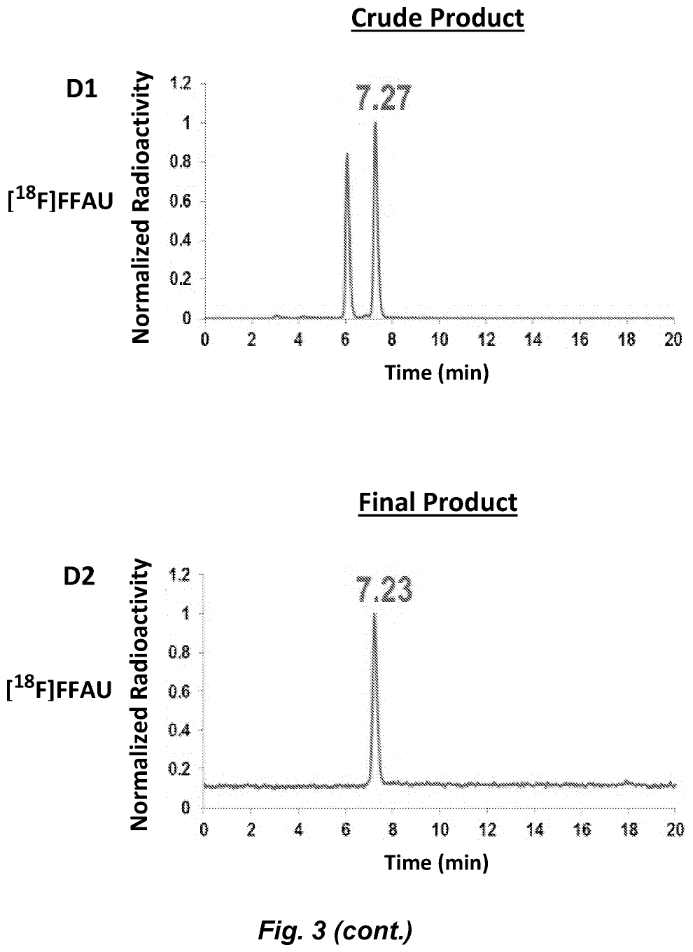

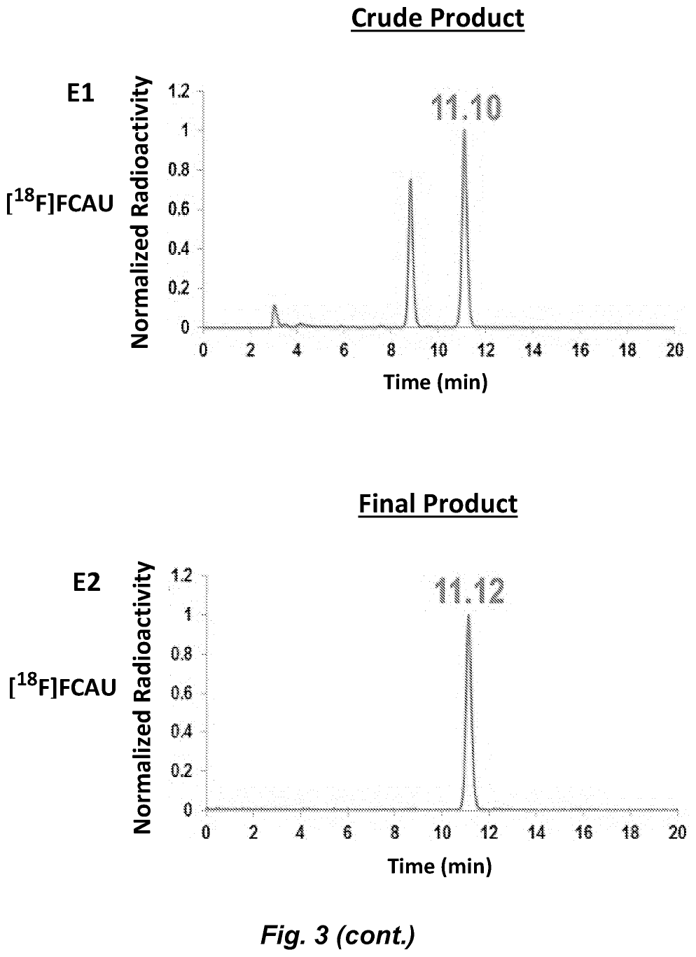

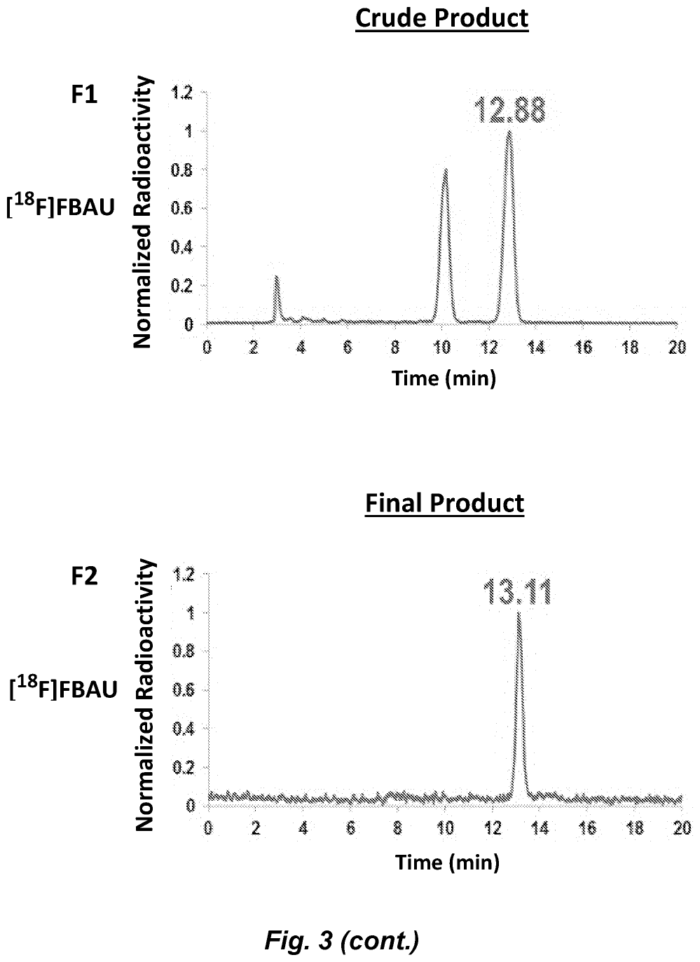

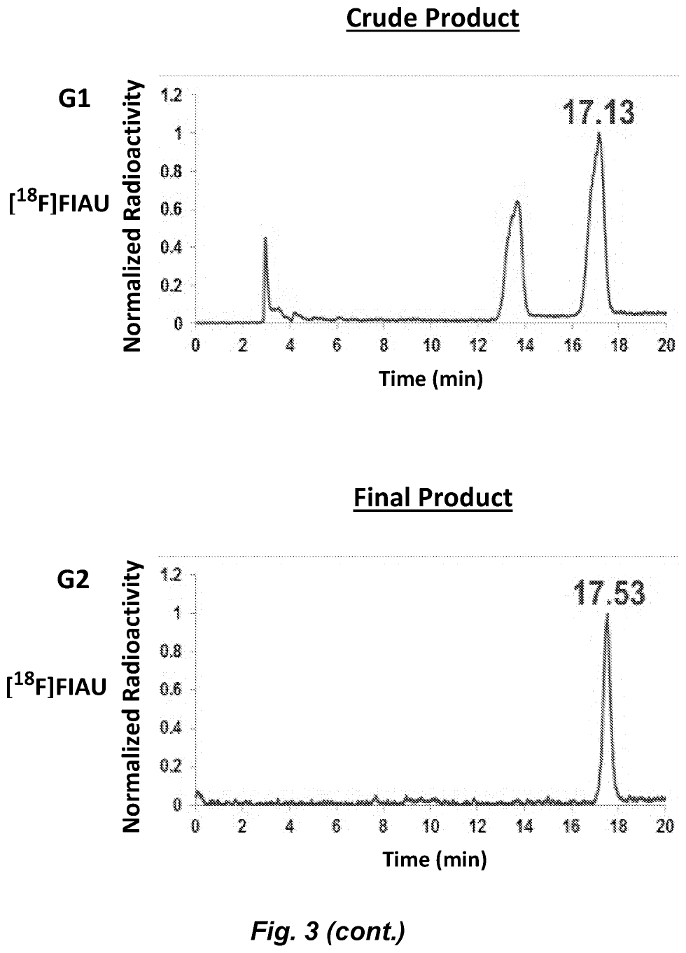

Next, 1,4-dioxane was applied to the radiosynthesis of other 2′-deoxy-2′-[¹⁸F]fluoro-5-substituted-1-β-D-arabinofuranosyluracil analogues, including [¹⁸F]FAU, [¹⁸F]FEAU, [¹⁸F]FFAU, [¹⁸F]FCAU, [¹⁸F]FBAU, and [¹⁸F]FIAU. Indeed, the results in FIG. 3 and Table 2 showed that our new method is quite versatile. The desired product (β-anomer) can be clearly identified in the crude product as shown in analytical HPLC profiles (FIG. 3). In addition, except for [¹⁸F]FCAU and [¹⁸F]FEAU, the RCY of the β-anomer is over 50% based on analytical HPLC, and the ratio of β/α anomers is greater than 1 in the radiosynthesis of [¹⁸F]FMAU, [¹⁸F]FFAU, [¹⁸F]FCAU, [¹⁸F]FBAU, and [¹⁸F]FIAU (Table 2). Notably, the RCY of the β-anomer in the current study using 1,4-dioxane is significantly higher than what was reported previously using DCE, suggesting that 1,4-dioxane as a coupling solvent is more effective in the radiosynthesis 2′-deoxy-2′-[¹⁸F]fluoro-5-substituted-1-β-D-arabinofuranosyluracil analogues.

TABLE 1
Solvent Effects on the Coupling of 2-Deoxy-2-
[¹⁸F]fluoro-1,3,5-tri-O-benzoyl-D-arabinofuranose
(¹⁸F-Labeled Sugar) and O,O′-Bis(trimethylsilyl)thymine
in the Radiosynthesis of [¹⁸F]FMAU (β-anomer)

| | | | Concen- | | | Ratio |
|---|---|---|---|---|---|---|
| | | Class of | tration | | % Yield | of |
| | | Residual | Limit | | of β- | anomers |
| Solvent | Polarity | Solvents^{a} | (ppm)^{a} | Toxicity | anomer^{b} | (β/α) |
| DMSO | Polar | 3 | 5000 | Low | ND^{c} | ND |
| DMF | Polar | 2 | 880 | Moderate | ND | ND |
| THF | Polar | 2 | 720 | Moderate | 5.37 | 2.86 |
| DCE | Nonpolar | 1 | 5 | High | 32.68 | 1.24 |
| 1,4- | Nonpolar | 2 | 380 | Moderate | 48.07 | 1.06 |
| Dioxane | | | | | | |

^{a}Data are cited from the United States Pharmacopeia (USP) General Chapter <467> Residual Solvents, Rev. 20190927.
^{b}Radiochemical yields (%) are reported based on the analysis of analytical HPLC.
^{c}ND; not detected.

TABLE 2
Radiochemical Yield (%) and Analytical HPLC Retention Time
of Crude and Final Product in the Radiosynthesis of [¹⁸F]FAU,
[¹⁸F]FMAU, [¹⁸F]FEAU, [¹⁸F]FFAU, [¹⁸F]FCAU,
[¹⁸F]FBAU, and [¹⁸F]FIAU Using 1,4-Dioxane as the Solvent

| | | | | HPLC Retention Time (min) | | |
|---|---|---|---|---|---|---|
| | % Radiochemical | | | Crude | | Final |
| | Yield^{a} | | Ratio of | Product | | Product |
| | α- | β- | anomers | α- | β- | β- |
| Radiotracer | anomer | anomer | (β/α) | anomer | anomer | anomer |
| [¹⁸F]FAU | 51.41 | 48.59 | 0.95 | 5.05 | 5.68 | 5.60 |
| [¹⁸F]FMAU | 44.34 | 55.66 | 1.26 | 7.44 | 8.93 | 9.10 |
| [¹⁸F]FEAU | 52.12 | 40.78 | 0.78 | 16.11 | 20.14 | 20.30 |
| [¹⁸F]FFAU | 44.08 | 54.82 | 1.24 | 6.07 | 7.27 | 7.23 |
| [¹⁸F]FCAU | 37.95 | 57.53 | 1.52 | 8.81 | 11.10 | 11.12 |
| [¹⁸F]FBAU | 39.44 | 55.69 | 1.41 | 10.19 | 12.88 | 13.11 |
| [¹⁸F]FIAU | 37.67 | 54.78 | 1.45 | 13.67 | 17.13 | 17.53 |

^{a}Radiochemical yield (%) is reported based on analytical HPLC.

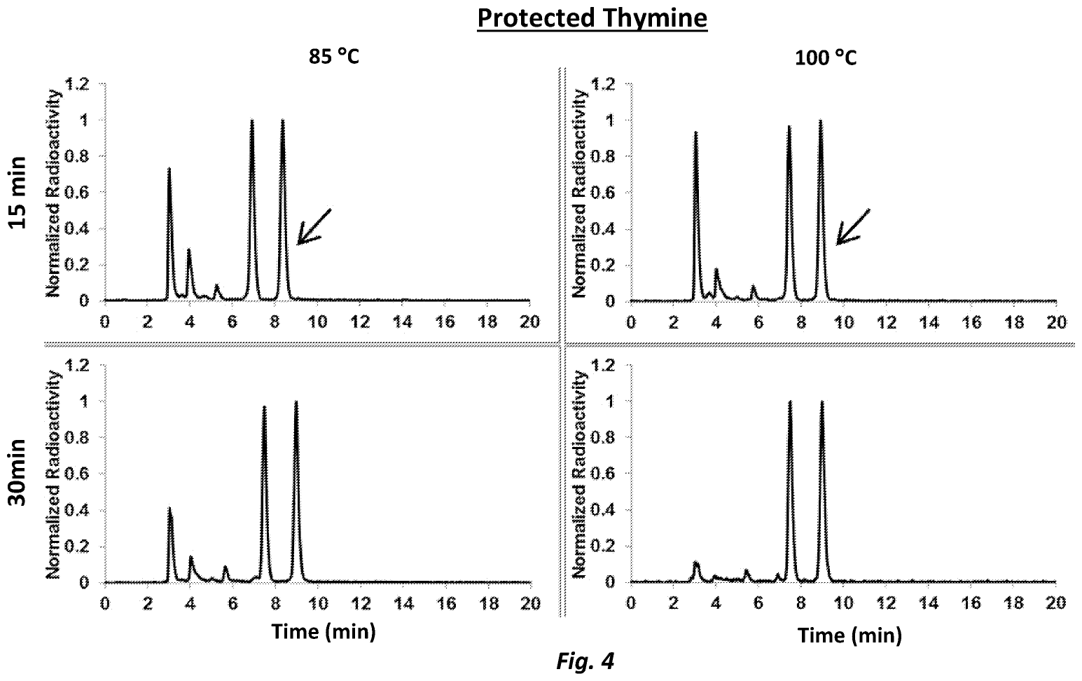

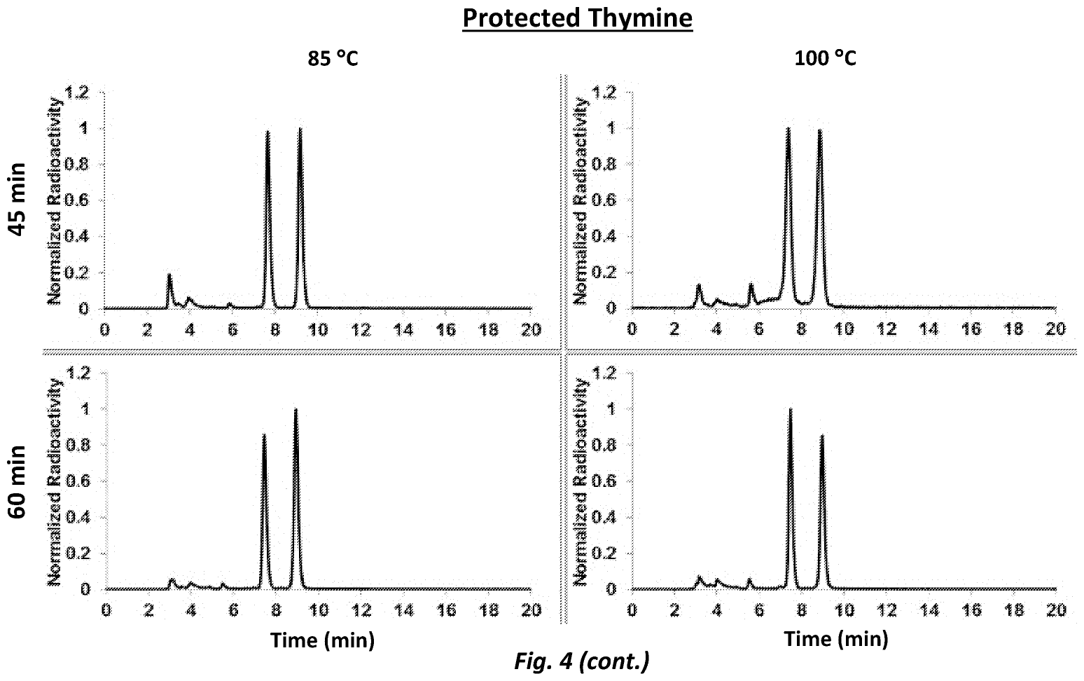

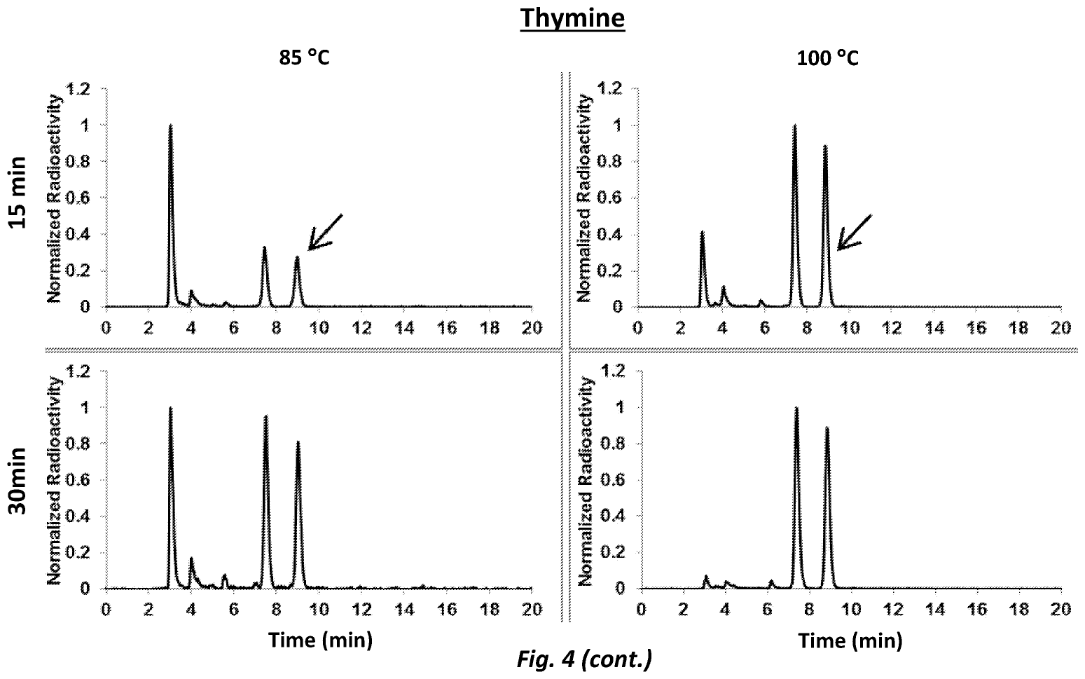

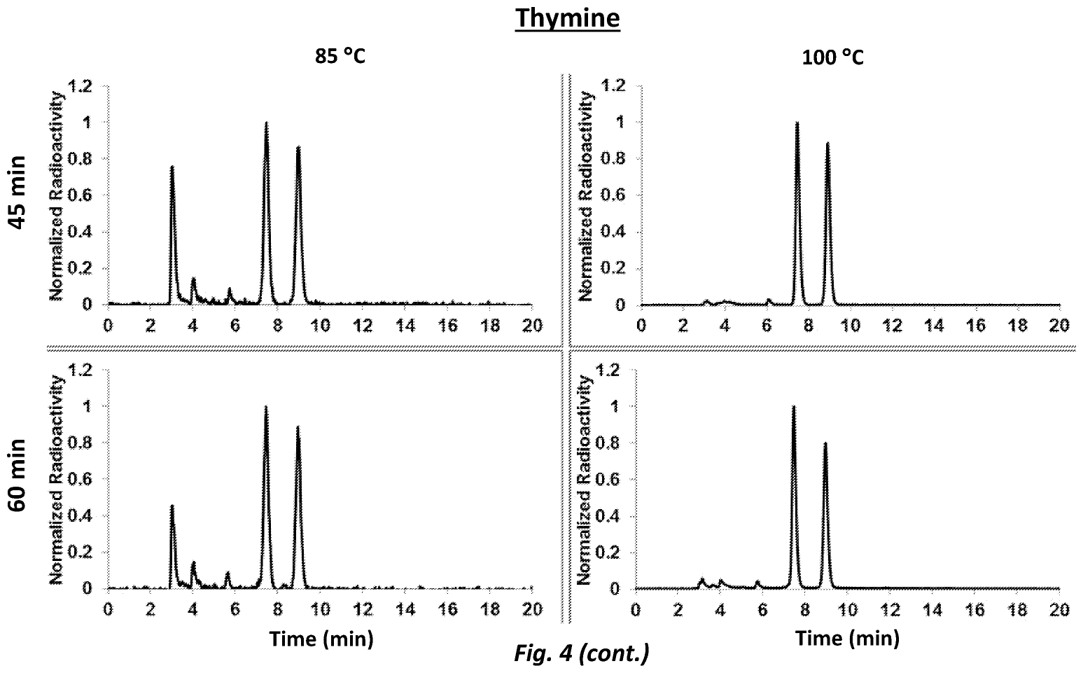

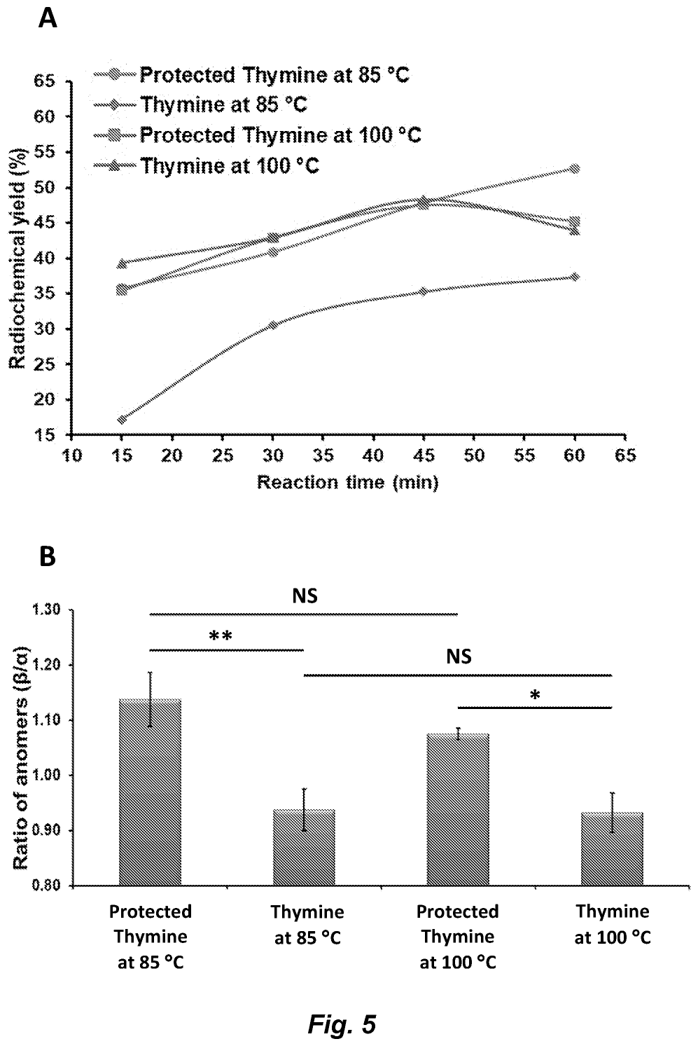

In order to improve the coupling efficiency and radiochemical yield, [¹⁸F]FMAU was utilized as an example to investigate the coupling step in the presence of 1,4-dioxane by changing various reaction factors, including reaction time (15, 30, 45, and 60 min), reaction temperature (85° C. and 100° C.), and the protected thymine vs. thymine. The results are shown in FIGS. 4 and 5. As a function of reaction time, the coupling efficiency is increased overall. For instance, in the case of the protected thymine and reaction temperature at 85° C., the RCY of the β-anomer was enhanced from 35.77% to 52.66% (FIG. 5A). Interestingly, the RCY of the β-anomer for both the protected thymine and thymine at 100° C. is decreased after heating the reaction 15 min longer (from 45 min to 60 min), indicating that appropriate reaction time is important for the coupling step at 100° C. In addition, fixing the reaction time at 45 min, no significant changes of the RCY of the β-anomer at 100° C. for the protected thymine and thymine were observed, suggesting that it may be not critical for the RCY at 100° C. using the protected thymine vs. thymine. However, a significant RCY improvement was observed at 85° C. for 45 min using the protected thymine vs. thymine (47.79% vs. 35.29%). Similarly, an enhanced RCY was yielded at 85° C. for 60 min using the protected thymine vs. thymine (52.66% vs. 37.36%). Furthermore, the ratio of β/α anomers was calculated based on the analysis of analytical HPLC for the coupling reaction at 60 min.

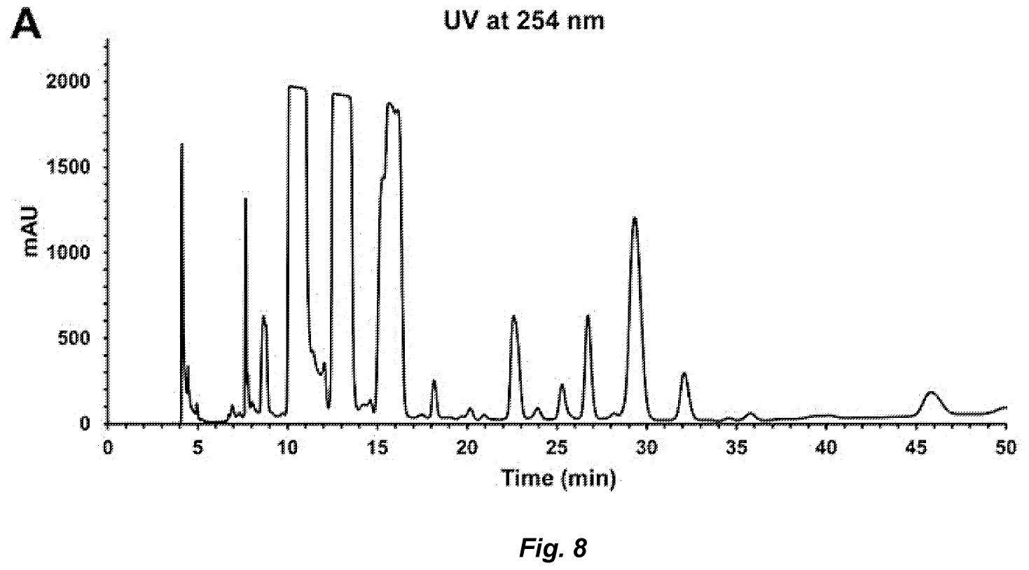

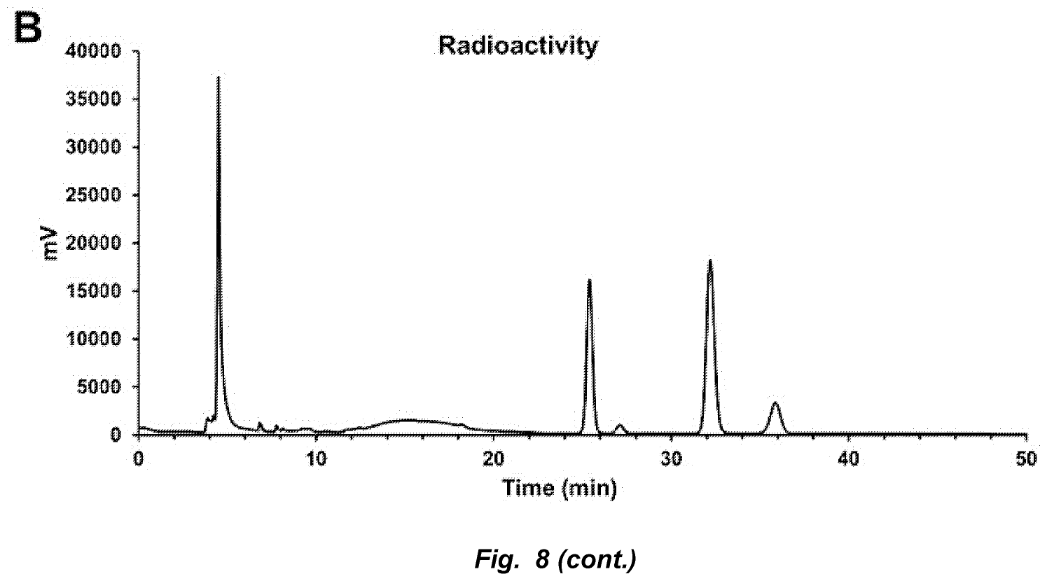

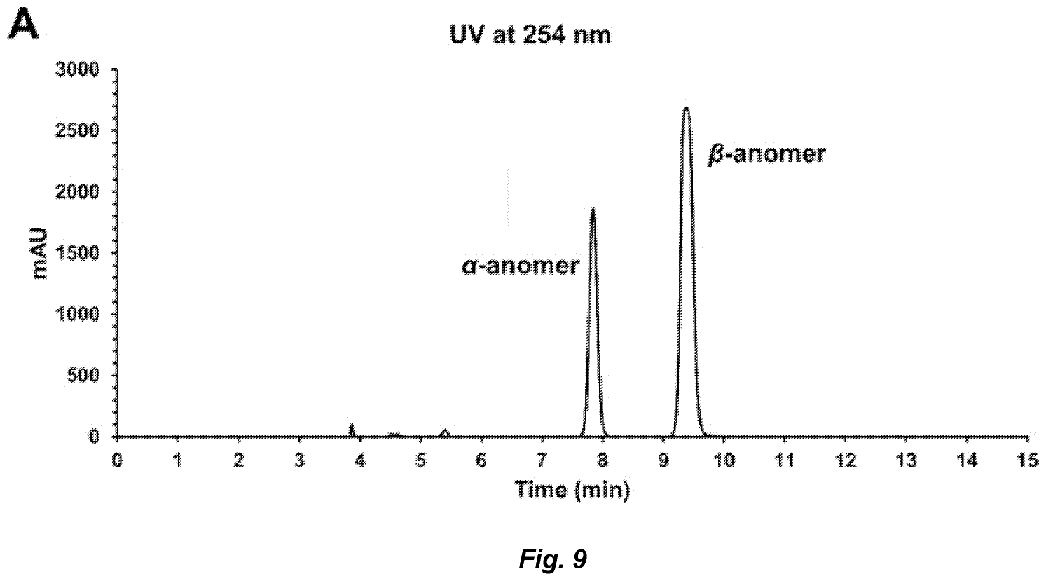

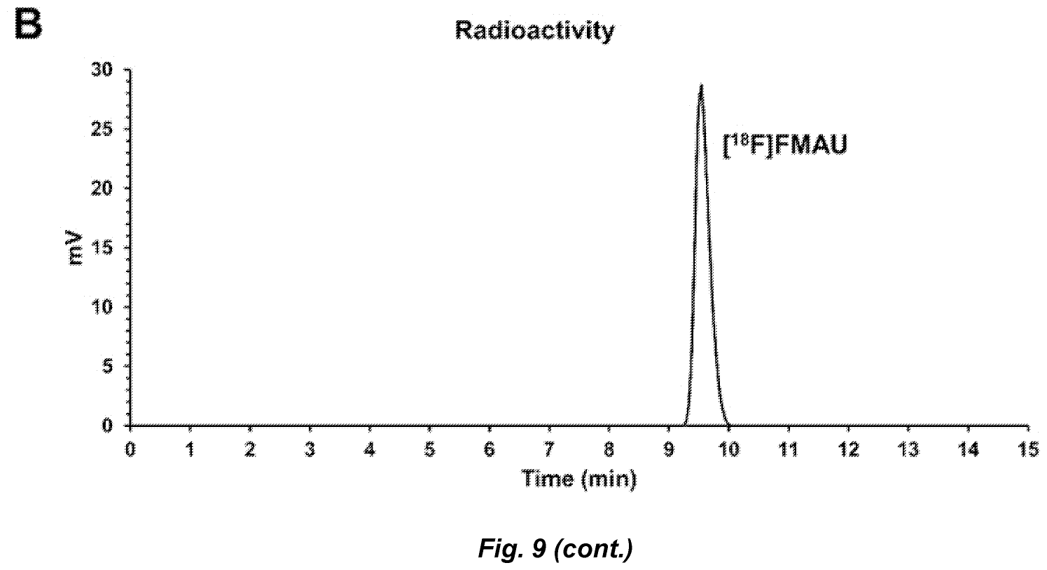

As shown in FIG. 5B, the ratio of β/α anomers for the protected thymine is significantly higher than that of thymine at 85° C. (1.14±0.05 vs. 0.94±0.04) and at 100° C. (1.08±0.01 vs. 0.93±0.04), demonstrating that using the protected thymine is critical to obtain a higher ratio of β/α anomers. Non-significant changes in the ratio of β/α anomers were observed for both the protected thymine and thymine at different temperatures (85° C. vs. 100° C.). Taken together, based on the results of the β-anomer RCY, the ratio of β/α anomers, and the length of reaction time, we determined that using the protected thymine and heating at 85° C. for 60 min is the best condition for the coupling step in the radiosynthesis of [¹⁸F]FMAU. The semi-preparative HPLC UV and radioactivity profiles of crude [¹⁸F]FMAU product using the newly developed method are presented in FIG. 8. The analytical HPLC UV and radioactivity for co-injection of cold authentic anomers (α- and β-anomer) and [¹⁸F]FMAU are displayed in FIG. 9.

### Quality Control for Process Validation Batches of [¹⁸F]FMAU

Three consecutive process validation batches of [¹⁸F]FMAU were prepared to fulfill the requirements of the Investigational New Drug (IND) application. Quality control testing of [¹⁸F]FMAU product was conducted according to the guidelines outlined in the USP and as described in the method section. Testing included visual inspection, pH, residual Kryptofix 222, chemical purity and radiochemical purity, specific activity, radionuclidic identity and purity, sterile filter integrity, bacterial endotoxin analysis, and sterility testing. Results for three process verification batches are reported in Table 3. All validation batches for process verification passed all required criteria for release. The results based on the new method of using 1,4-dioxne for [¹⁸F]FMAU manufacture are satisfied with the submission of the IND application.

TABLE 3
Quality Control Data for Process Verification Batches of [¹⁸F]FMAU

| QC Test | Release Criteria | | Batch 1 | Batch 2 | Batch 3 |
|---|---|---|---|---|---|
| Radioactivity | 1-75 | mCi/mL | 18.3838 | 8.3224 | 17.1949 |
| concentration at | | | | | |
| end of synthesis | | | | | |
| (mCi/mL) | | | | | |
| Final product | Clear, Colorless, and | | Pass | Pass | Pass |
| appearance | free of particulates | | | | |
| Filter membrane | ≥50 | psi | 63 | 63 | 62 |
| integrity | | | | | |
| (bubble-point | | | | | |
| test) (psi) | | | | | |
| Kryptofix Test | ≤50 | μg/mL | Pass | Pass | Pass |
| Radiochemical | Within 0.5 min of the | | Pass | Pass | Pass |
| identity (HPLC) | reference standard | | Standard: | Standard: | Standard: |
| | retention time | | 9.927 min | 9.947 min | 9.953 min |
| | | | Sample: | Sample: | Sample: |
| | | | 10.002 min | 10.083 min | 10.102 min |
| Radiochemical | ≥95% | | 99.40 | 100.00 | 99.44 |
| purity (HPLC) | | | | | |
| (%) | | | | | |
| Chemical purity | ≤8.33 | μg/mL | 1.6900 | 2.3075 | 4.3006 |
| (FMAU mass, | | | | | |
| μg/mL) | | | | | |
| Total impurity | <3.6 | μg/dose | 0.5775 | 1.5320 | 0.4868 |

| | | | | |
|---|---|---|---|---|
| (non-FMAU | | | | |
| impurities)^{a} | | | | |
| Residual | Methanol: ≤3000 ppm | Pass | Pass | Pass |
| solvents (GC) | Acetonitrile: ≤410 ppm | | | |
| (ppm) | 1,4-Dioxane: ≤380 ppm | | | |
| Radionuclidic | Between 105 and 115 | 109.5637 | 110.1338 | 109.3802 |
| identity (half- | min | | | |
| life) | | | | |
| Radionuclidic | Peak value is present | 511.7 | 511.7 | 511.7 |
| purity (KeV) | between 501 and 521 | | | |
| | KeV | | | |
| Final product pH | 4.0-7.5 | 5.0 | 5.0 | 5.0 |
| Bacterial | ≤17.5 EU/mL with | <5 EU/mL | <5 EU/mL | <5 EU/mL |
| endotoxin test | maximum dose volume | | | |
| | 10 mL | | | |
| 14-Day sterility | Absence of microbials | Pass | Pass | Pass |
| test | after 14-day incubation | | | |
| | in two kinds of media | | | |

^{a}Total impurity value includes only the un-identified impurities, i.e. non-FMAU impurities.

### Partition Coefficient

The hydrophilicity of PET tracers was examined by measuring the 1-octanol/PBS partition coefficient value as expressed as Log P. The Log P values of [¹⁸F]FAU, [¹⁸F]FMAU, [¹⁸F]FEAU, [¹⁸F]FFAU, [¹⁸F]FCAU, [¹⁸F]FBAU, and [¹⁸F]FIAU were determined to be −0.943±0.041, −0.577±0.003, −0.077±0.018, −0.952±0.023, −0.477±0.030, −0.367±0.025, and −0.108±0.013, respectively (Table 4). The Log P values suggest that the hydrophilicity is gradually reduced when the 5-hydrogen of 2′-deoxy-2′-[¹⁸F]fluoro-1-β-D-arabinofuranosyluracil is substituted by fluoro, methyl, chloro, bromo, iodo, and ethyl groups, respectively. The hydrophilicity of these analogues determined by Log P showed similar pattern in general as appeared at the retention times on the analytical HPLC (FIG. 3 and Table 2).

TABLE 4
Measured 1-Octanol/PBS Partition Coefficients and
Log P Values of [¹⁸F]FAU, [¹⁸F]FMAU, [¹⁸F]FEAU,
[¹⁸F]FFAU, [¹⁸F]FCAU, [¹⁸F]FBAU, and [¹⁸F]FIAU

| Radiotracer | Partition coefficients of 1-octanol/PBS^{a} | Log P |
|---|---|---|
| [¹⁸F]FAU | 0.114 ± 0.011 | −0.943 ± 0.041 |
| [¹⁸F]FMAU | 0.265 ± 0.002 | −0.577 ± 0.003 |
| [¹⁸F]FEAU | 0.837 ± 0.035 | −0.077 ± 0.018 |
| [¹⁸F]FFAU | 0.113 ± 0.006 | −0.952 ± 0.023 |
| [¹⁸F]FCAU | 0.334 ± 0.022 | −0.477 ± 0.030 |
| [¹⁸F]FBAU | 0.430 ± 0.024 | −0.367 ± 0.025 |
| [¹⁸F]FIAU | 0.780 ± 0.024 | −0.108 ± 0.013 |

^{a}Measurements were carried out in quintuplicate for each tracer.

### PET Imaging

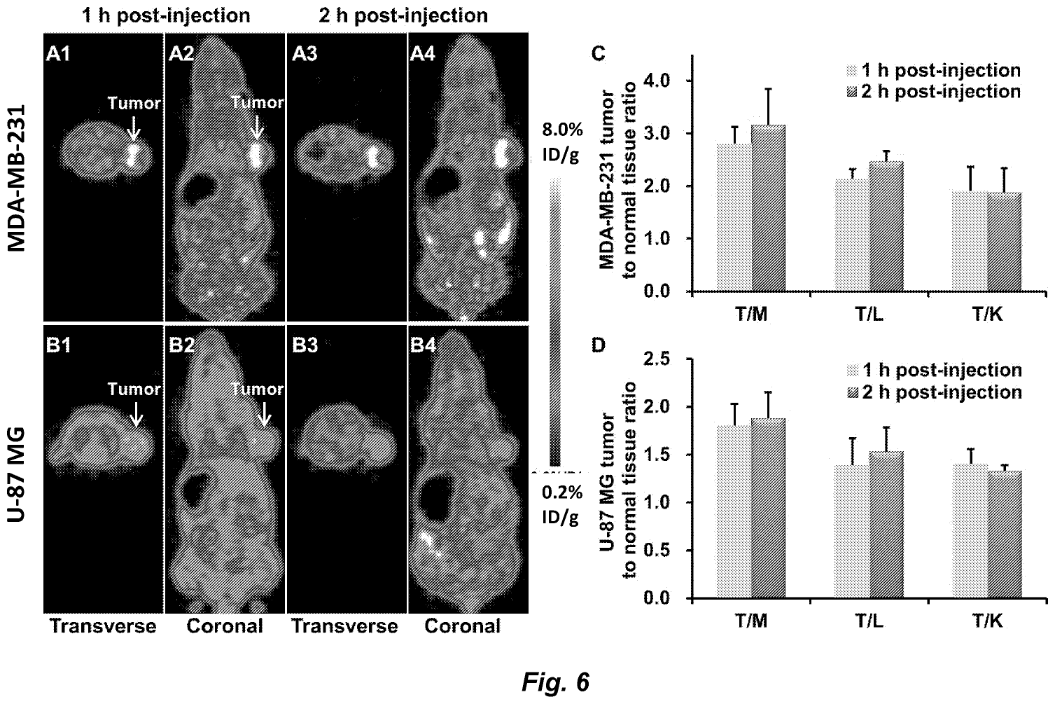

Next, tumor PET imaging of [¹⁸F]FMAU in animals. Two aggressive tumor cell lines were selected for this process, MDA-MB-231, a triple-negative breast cancer cell line, and U-87 MG glioblastoma cell line, to establish tumor xenografts in mice. After the intravenous injection of [¹⁸F]FMAU at 1 h and 2 h, the mice (n=3/group) were scanned through a microPET imaging system. The representative decay-corrected transverse and coronal sections that contained the tumors at 1 h and 2 h post-injection (p.i.) are displayed in FIG. 6, panels A1-A4 (MDA-MB-231 tumor model) and panels B1-B4 (U-87 MG tumor model). For microPET scans, radioactivity accumulations in tumors and major tissues/organs were quantified by calculating the ROIs that comprised the entire organ on the coronal images.

For the MDA-MB-231 tumor model, tumor uptake of [¹⁸F]FMAU was calculated to be 6.4±0.4 and 7.2±0.6% ID/g at 1 h and 2 h p.i., respectively. The ratio of MDA-MB-231 tumor uptake to muscle, liver, and kidney uptake was calculated to be 2.8±0.3, 2.1±0.2, and 1.9±0.5 (at 1 h p.i.), and 3.2±0.7, 2.5±0.2, and 1.9±0.5 (at 2 h p.i.), respectively. For the U-87 MG tumor model, tumor uptake of [¹⁸F]FMAU was calculated to be 6.0±0.2 and 5.6±0.4% ID/g at 1 h and 2 h p.i., respectively. The ratio of U-87 MG tumor uptake to muscle, liver, and kidney uptake was calculated to be 1.8±0.2, 1.4±0.3, and 1.4±0.2 (at 1 h p.i.), and 1.9±0.3, 1.5±0.3, and 1.3±0.1 (at 2 h p.i.), respectively. At 1 h vs. 2 h p.i., non-significant changes were observed for the ratio of T/M, T/L, and T/K in both tumor models. At all imaging time points, tumors were clearly visible with good contrast to the background. We believe that the newly developed radiosynthesis method of [¹⁸F]FMAU and its analogues will facilitate future investigations in both pre-clinical and clinical studies.

5-Substituted 2′-deoxy-2′-[¹⁸F]fluoro-arabino-furanosyluracil analogues were synthesized in excellent radiochemical purity using an improved synthesis method. 1,4-Dioxane is a less-toxic alternative to DCE that also provides better radiosynthetic yields. The use of a less toxic solvent allows for the translation of the improved approach to clinical production. This new method is versatile, which permits a broad range of use for ¹⁸F-labeling of other nucleoside analogues.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Material and Methods

### Materials

2-O-(trifluoromethanesulfonyl)-1,3,5-tri-O-benzoyl-α-D-ribofuranose was either synthesized in accordance with the reported procedure³⁵ or obtained from ABX advanced biochemical compounds GmbH (Germany). [¹⁸O]H₂O was purchased from Huayi Isotopes Co. All other chemicals and solvents were obtained from Sigma-Aldrich. 1,4-Dioxane (anhydrous, 99.8%) was tested for peroxide formation prior to use after opening the bottle. The ion exchange cartridges were obtained from ABX advanced biochemical compounds GmbH (Germany).

### HPLC Methods

Analytical and semi-preparative reversed phase high-performance liquid chromatography (HPLC) were carried out using two Thermo Scientific UltiMate 3000 HPLC systems. Semi-preparative HPLC was performed using a Phenomenex Luna C18(2) reversed phase column (5 μm, 250×10 mm). The flow rate was 3.5 mL/min with the isocratic mobile phase of 4% acetonitrile in water. The UV absorbance was recorded at 254 nm. Analytical HPLC was accomplished using a Phenomenex Luna C18(2) reversed phase column (5 μm, 250×4.6 mm). The flow rate was 1 mL/min with the isocratic mobile phase of 8% acetonitrile in water with 0.1% trifluoroacetic acid (TFA). The UV absorbance was recorded at 254 nm. The Model 101 and Model 105 radiodetectors (Carroll & Ramsey Associates, Berkeley, CA) were used for the semi-preparative and analytical HPLC system, respectively.

### Radiosynthesis of [¹⁸F]FMAU and its Analogues

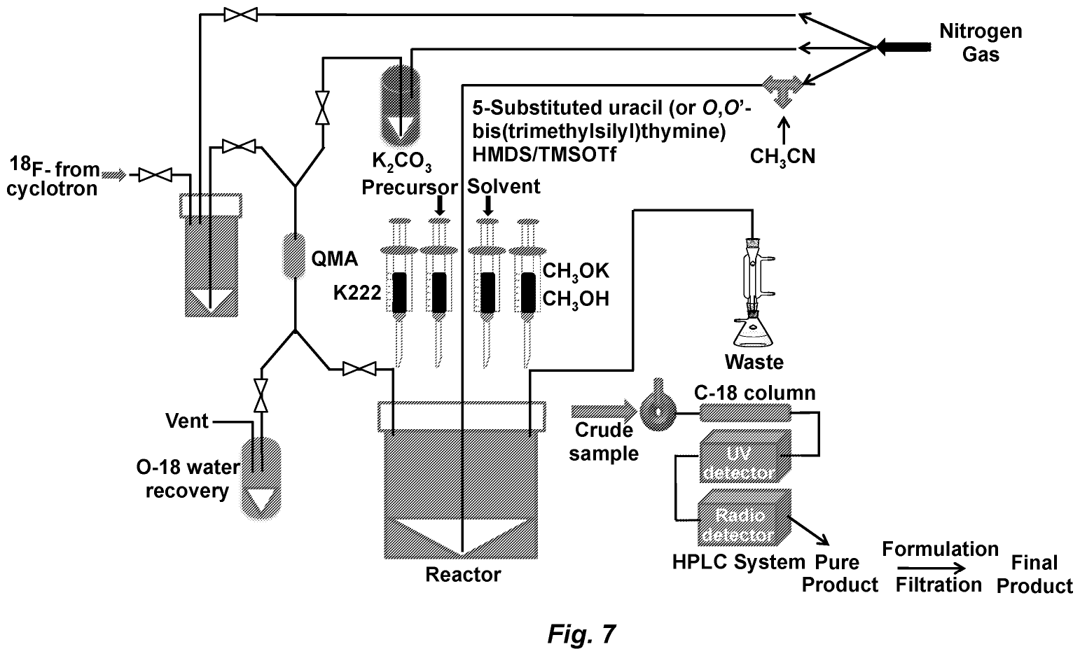

Radiosyntheses of [¹⁸F]FMAU and its analogues were carried out in a semi-automatic synthesis module (FIG. 7) and as generally described in U.S. Pat. No. 8,912,319 (Li et al.), which is incorporated herein by reference in its entirety, and as modified below. The [¹⁸F]fluoride ion was generated by the nuclear reaction [¹⁸O] (p, n) [¹⁸F] in a GE PETtrace 800 cyclotron. [¹⁸F]fluoride ion in [¹⁸O]water was transferred through a pre-conditioned QMA cartridge, and the retained [¹⁸F]fluoride was eluted to a V-vial with a potassium carbonate solution (7.5 mg in 650 μL of deionized water). Kryptofix 222 solution (15.0 mg in 1.0 mL of anhydrous acetonitrile) was added to the V-vial, and the mixture solution was dried at 100° C. with nitrogen flow. Additional anhydrous acetonitrile was added to the V-vial and the reaction solution was azeotropically dried. The precursor 2-O-(trifluoromethanesulfonyl)-1,3,5-tri-O-benzoyl-α-D-ribofuranose solution (10.0 mg in 0.8 mL of anhydrous acetonitrile) was added to the dried ¹⁸F ion and heated at 85° C. for 20 min. Afterwards, O,O′-bis(trimethylsilyl)thymine (20 mg) or other 5-substituted uracil analogues, 200 μL of HMDS, 300 μL of 1,4-dioxane, and 150 μL of TMSOTf were added to the V-vial. The reaction solution was heated at 85° C. or 100° C. for various reaction times (15, 30, 45, and 60 min). After removing solvent, 400 UL of potassium methoxide solution (25% in methanol) and 400 μL of methanol were added. The mixture was heated at 85° C. for 5 min. After removing methanol, 6 N HCl was added to the reaction mixture. The crude reaction mixture was analyzed by analytical HPLC and purified by semi-preparative HPLC. The chemical purity and radiochemical purity of final product were analyzed by HPLC. For the process validation batches of [¹⁸F]FMAU, 0,0′-bis(trimethylsilyl)thymine (20 mg), 200 μL of HMDS, 300 μL of 1,4-dioxane, and 150 μL of TMSOTf were used in the coupling step.

### Quality Control for Process Validation Batches of [¹⁸F]FMAU

All of the analytical test procedures were performed using high-quality solvents (≥99.5% purity), reagents, and materials which were carefully logged in, controlled, and verified in the same manner as the reagents for the manufacturing process. The drug product was assayed for total radioactivity using a qualified dose calibrator. The physical appearance of the drug product in the vial was done by careful visual inspection under enough light. The final drug product in the vial must be clear and colorless without any visible particulates. Two samples totaling nominally ≥0.2 mL/sample are removed for quality control and sterility test. The integrity of the sterilizing filter was tested. The filter was tested with increasing pressure applied by a calibrated gauge. The bubble point result must exceed the pressure of the manufacturer's specification to confirm filter integrity. The Kryptofix test was performed to demonstrate that the final product sample spot must show less intensity than the spot from the Kryptofix standard solution with a concentration of 50 μg/mL.

The retention time of standard FMAU was obtained using a certified standard produced by ABX advanced biochemical compounds GmbH (Germany). The radiochemical identity specification requires the agreement of drug product and standard retention time within 0.5 min. The specification for the radiochemical purity was set up to be equal to or greater than 95%. The identity of [¹⁸F]FMAU was validated by comparing the retention time of the nonradioactive FMAU standard and the [¹⁸F]FMAU drug product. HPLC chromatography analysis was also applied to analyze chemical purity for the drug product. The specification of FMAU concentration was set up to be equal to or less than 8.33 μg/mL based on our previous experience with [¹¹C]FMAU in non-human primates and humans. The amount of FMAU was calculated based on the FMAU UV peak area and the calibration curve. The Total Impurity in the [¹⁸F]FMAU drug product was set up to be less than 3.6 μg/dose. This value includes only the un-identified impurities, i.e. non-FMAU impurities.

Residual solvent levels were determined using gas chromatography (GC). Methanol, acetonitrile, and 1,4-dioxane were used for the production of [¹⁸F]FMAU and thus are potential residual solvent impurities. The permissible level of methanol, acetonitrile, and 1,4-dioxane in the final product must be equal to or less than 3000 ppm, 410 ppm, and 380 ppm, respectively as stated in the USP <467> residual solvent limits.

The radionuclidic identity of the final product was determined by measuring the half-life of the radionuclide in order to assure it is [¹⁸F]fluorine. This test was used to determine the identity of the radioactive nuclide of [¹⁸F]fluorine in the sample of the final product. A sample was allowed to decay for a predetermined time and beginning and ending radioactivity measurements were compared and half-life calculated. The expected half-life of ¹⁸F is 109.77 min. In the test to show radionuclidic identity, the half-life test result for ¹⁸F must be between 105 and 115 min. The radionuclidic purity of the final product was determined by multi-channel analysis (MCA). Photopeak energy for radioactive decay of [¹⁸F]fluorine is 511 KeV. Photopeak of the sample associated with radioisotopic decay must be observed at the peak between 501 KeV and 521 KeV and possibly at 1.022 MeV (sum peak).

The specification of pH was set up to the range of 4.0-7.5. Bacterial endotoxin levels were tested using the Charles River Endosafe PTS system. The releasing specification for the bacterial endotoxin level is ≤17.5 EU/mL with a maximum injection volume of 6 mL. The 14-day sterility was tested using the direct inoculation method where a sample was inoculated into two types of media within 30 hours after synthesis of the drug product.

### Partition Coefficient

The octanol-PBS partition coefficient was measured at room temperature according to the previously reported procedure, and the value was designated as Log P.^{37, 38} In brief, [¹⁸F]FMAU or other 5-substituted thymidine analogues (370 KBq) in 5 μL of phosphate-buffered saline (PBS) (pH=7.4) was added to an Eppendorf tube including 500 μL of PBS (pH 7.4) and 500 μL of 1-octanol. The mixture was vortexed for 5 min and then centrifuged (12,500 rpm) for 8 min. The PBS and 1-octanol layers (200 μL of each layer) were pipetted into gamma-counter test tubes, respectively. The radioactivity was determined using a PerkinElmer 2480 WIZARD² automatic gamma counter (PerkinElmer Inc., Waltham, MA). The partition coefficients of 1-octanol-to-PBS were calculated as P=(organic-phase cpm−background cpm)/(aqueous-phase cpm−background cpm), and the values were expressed as Log P. Measurements were carried out in quintuplicate for each radiotracer.

### Cell Culture

Both MDA-MB-231 human adenocarcinoma and U-87 MG human glioblastoma cell lines were purchased from American Type Culture Collection (Manassas, VA, USA). Tumor cells were cultured in Dulbecco's Modified Eagle Medium (DMEM) supplemented with 10% fetal bovine serum at 37° C. in a humidified incubator containing 5% CO₂.

### Animal Tumor Models

All animal studies were approved by the Institutional Animal Care and Use Committee of University of Southern California. Both MDA-MB-231 and U-87 MG tumor xenograft models (n=3/group) were generated by subcutaneous injection of 5×10⁶ tumor cells into the front right flank of female athymic nude mice (4-6 weeks old) purchased from Envigo Inc., Indianapolis, IN. The tumors were permitted to grow 2-4 weeks until approximate 0.6-0.8 cm³ in volume.

### MicroPET Imaging

MicroPET scans were carried out using a rodent scanner (Siemens Inveon microPET scanner, Siemens Medical Solutions). About 7.4 MBq (200 μCi) of [¹⁸F]FMAU was injected through the tail vein under isoflurane anesthesia condition. Five-minute static scans were obtained at 60- and 120-min post-injection (p.i.). The 3D-OSEM algorithm was applied for image reconstruction. For each microPET scan, the regions of interest (ROIs) were drawn over tumor, muscle, liver, and kidneys on the decay-corrected whole-body coronal images. The tumor-to-muscle (T/M), tumor-to-liver (T/L), and tumor-to-kidney (T/K) ratios were then calculated.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method of synthesizing 2′-deoxy-2′-[¹⁸F]-fluoro-5-substituted-1-β-D-arabinofuranosyl-uracil or cytosine compounds in one-pot comprising:
a) radiolabeling a precursor sugar with ¹⁸F;
b) contacting the ¹⁸F radiolabeled sugar with a silylated uracil or cytosine in the presence of 1,4-dioxane, trimethylsilyl trifluoromethylsulfonate (TMSOTf), and hexamethyldisilazane (HMDS);
c) incubating the components in step (b) under conditions that allow for conjugation of the ¹⁸F radiolabeled sugar and the silylated uracil or cytosine; and
d) removing the protecting groups of the components in step (c);
wherein steps a) to d) are performed in one-pot.

2. The method according to claim 1 wherein the 2′-deoxy-2′-[¹⁸F]-fluoro-5-substituted-1-β-D-arabinofuranosyluracil is 2′-deoxy-2′-[¹⁸F]fluoro-5-methyl-1-β-D-arabinofuranosyl-uracil ([¹⁸F]FMAU).

3. The method according to claim 1 wherein the [¹⁸F]-labeled 2′-deoxy-arabino 5-substituted or unsubstituted uracil or cytosine nucleoside is one or more of 2′-Deoxy-2′-[¹⁸F]fluoro-5-methyl-1-β-d-arabinofuranosyluracil ([¹⁸F]FMAU), 2′-fluoro-5-ethyl-1-β-d-arabinofuranosyluracil ([¹⁸F]FEAU), 2′-deoxy-2′-fluoro-5-fluoro-1-β-d-arabinofuranosyluracil ([¹⁸F]FFAU), 1-(2-deoxy-2-fluoro-β-d-arabinofuranosyl)-5-chlorouracil ([¹⁸F]FCAU), 1-(2-deoxy-2-fluoro-β-d-arabinofuranosyl)-5-bromouracil ([¹⁸F]FBAU), 1-(2-deoxy-2-fluoro-β-d-arabinofuranosyl)uracil ([¹⁸F]FAU), 2′-fluoro-2′-deoxy-1-β-d-arabinofuranosyl-5-iodouracil ([¹⁸F]FIAU), 1-(2-deoxy-2-fluoro-β-d-arabinofuranosyl)cytosine ([¹⁸F]FAC), 2′-deoxy-2′-fluoro-5-methyl-1-β-d-arabinofuranosylcytosine ([¹⁸F]FMAC), 2′-fluoro-5-ethyl-1-β-d-arabinofuranosyl-cytosine ([¹⁸F]FEAC), 2′-Deoxy-2′-fluoro-5-fluoro-1-β-d-arabinofuranosyluracil ([¹⁸F]FFAC), 1-(2-deoxy-2-fluoro-β-d-arabinofuranosyl)-5-chlorocytosine ([¹⁸F]FCAC), 1-(2-deoxy-2-fluoro-β-d-arabinofuranosyl)-5-bromocytosine ([¹⁸F]FBAC), and 2′-deoxy-2′-fluoro-5-hydroxymethyl-1-β-d-arabino-furanosylcytosine ([¹⁸F]FHMAC).

4. The method according to claim 1 wherein the contacting of step (b) further comprises one or more organic solvents, inorganic solvents, or a combination thereof.

5. The method according to claim 1 wherein the incubating of step (c) is carried out at about 70° C. to about 110° C.

6. The method according to claim 5 wherein the incubating of step (c) is carried out at about 75° C. to about 95° C.

7. The method according to claim 1 wherein the incubating step (c) is carried out for about 5 minutes to about 120 minutes.

8. The method according to claim 7 wherein the incubating step (c) is carried out for about 40 minutes to about 80 minutes.

9. The method according to claim 1 wherein an amount of residual methanol is 3000 parts per million (PPM) or less, acetonitrile is 410 PPM or less, and 1,4-Dioxane is 380 PPM or less.

10. A method for the fully automated synthesis of [¹⁸F]FMAU comprising the method of claim 1 wherein synthesis takes place in a fully automated cGMP-compliant radiosynthesis module.

11. A method of synthesizing an [¹⁸F]-labeled 2′-deoxy-arabino 5-substituted or unsubstituted uracil or cytosine nucleoside in one-pot comprising:
a) radiolabeling a precursor sugar with ¹⁸F;
b) contacting the ¹⁸F radiolabeled sugar with a silylated uracil or cytosine in the presence of 1,4-dioxane, trimethylsilyl trifluoromethylsulfonate (TMSOTf), and hexamethyldisilazane (HMDS);
c) incubating the components in step (b) under conditions that allow for conjugation of the ¹⁸F radiolabeled sugar and the silylated uracil or cytosine derivatives; and
d) removing the protecting groups of the components in step (c);
wherein steps a) to d) are performed in one-pot.

12. The method according to claim 11 wherein the [¹⁸F]-labeled 2′-deoxy-arabino 5-substituted or unsubstituted uracil or cytosine nucleoside is one or more of 2′-Deoxy-2′-[¹⁸F]fluoro-5-methyl-1-β-d-arabinofuranosyluracil ([¹⁸F]FMAU), 2′-fluoro-5-ethyl-1-β-D-arabinofuranosyluracil ([¹⁸F]FEAU), 2′-deoxy-2′-fluoro-5-fluoro-1-β-D-arabinofuranosyluracil ([¹⁸F]FFAU), 1-(2-deoxy-2-fluoro-β-D-arabinofuranosyl)-5-chlorouracil ([¹⁸F]FCAU), 1-(2-deoxy-2-fluoro-β-D-arabinofuranosyl)-5-bromouracil ([¹⁸F]FBAU), 1-(2-deoxy-2-fluoro-β-D-arabinofuranosyl)uracil ([¹⁸F]FAU), 2′-fluoro-2′-deoxy-1-β-D-arabinofuranosyl-5-iodouracil ([¹⁸F]FIAU), 1-(2-deoxy-2-fluoro-β-D-arabinofuranosyl)cytosine ([¹⁸F]FAC), 2′-deoxy-2′-fluoro-5-methyl-1-β-D-arabinofuranosylcytosine ([¹⁸F]FMAC), 2′-fluoro-5-ethyl-1-β-D-arabinofuranosyl-cytosine ([¹⁸F]FEAC), 2′-Deoxy-2′-fluoro-5-fluoro-1-β-D-arabinofuranosyluracil ([¹⁸F]FFAC), 1-(2-deoxy-2-fluoro-β-D-arabinofuranosyl)-5-chlorocytosine ([¹⁸F]FCAC), 1-(2-deoxy-2-fluoro-β-D-arabinofuranosyl)-5-bromocytosine ([¹⁸F]FBAC), and 2′-deoxy-2′-fluoro-5-hydroxymethyl-1-β-D-arabino-furanosylcytosine ([¹⁸F]FHMAC).

13. A method for fully automated synthesis [¹⁸F]-labeled thymidine or cytidine analogues comprising the method of claim 11, wherein the synthesis is fully automated using a cGMP-compliant radiosynthesis module.

14. A method of synthesizing a 2′-deoxy-2′-[¹⁸F]-fluoro-5-substituted-1-β-D-arabinofuranosyl-uracil or cytosine compound comprising:
a) radiolabeling a precursor sugar with ¹⁸F;
b) filtering the ¹⁸F radio labeled sugar produced in step (a) through a cartridge;
c) contacting the ¹⁸F radiolabeled sugar with a silylated uracil or cytosine in the presence of a Friedel-Crafts catalyst and 1,4-dioxane;
d) incubating the components in step (c) under conditions that allow for conjugation of the ¹⁸F radiolabeled sugar and the silylated uracil or cytosine; and
e) incubating the components in step (d) under conditions that allow for removal of the protecting groups of the components in step (d), thereby removing the protecting groups of the components in step (d).
